(19) United States
(12) Patent Application Publication
(10) Pub. No.: US 2003/0091555 A1
(43) Pub. Date: May 15, 2003

(54) Bactericidal composition containing peptide and chelating agent

(75) Inventor: Shigeru Oita, Zentsuji-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: NATIONAL AGRICULTURAL RESEARCH ORGANIZATION, Tsukuba-shi (JP)

(21) Appl. No.: 10/067,124

(22) Filed: Feb. 4, 2002

(30) Foreign Application Priority Data
Aug. 22, 2001 (JP) 2001-251048

(51) Int. Cl. A61K 38/48; A61K 31/195

(52) U.S. Cl. 424/94.63; 514/564

(57) ABSTRACT

A bactericidal composition is provided, which comprises as effective ingredients (a) at least one substance selected from the group consisting of ethylenediaminetetraacetic acid and metal salts thereof and (b) at least one substance selected from the group consisting of alpha-type thionin and beta-type thionin. The bactericidal composition, for example, has the effect of sterilizing food poisoning bacteria at a low concentration and is highly safe.

### FIELD OF THE INVENTION

The present invention relates to a bactericidal composition containing a peptide and a chelating agent, and more particularly, to a bactericidal composition containing a chelating agent such as ethylenediaminetetraacetic acid and an antibacterial peptide derived from barley as effective ingredients.

### BACKGROUND OF THE INVENTION

In Japan, the bacteria that cause food poisoning most frequently include Salmonella sp. and Vibrio parahaemolyticus. In recent years, food poisoning due to pathogenic Escherichia coli O-157 is increasing trend. Food poisoning of rice products due to Bacillus cereus occurs every year though in few cases.

To sterilize these food poisoning bacteria contaminating various foods is effective for preventing the food poisoning.

However, consumers tend to dislike chemically synthesized food preservatives and bactericides. Therefore, various efforts have been made to contrive an efficient mechanism that they can act at low concentrations and to control the bacteria by use of a natural substance derived from a plant.

The antibacterial substance derived from a plant includes allyl isothiocyanate, which is a component of mustard and horseradish (Journal of the Japanese Society for Food Science and Technology, Vol. 37, pages 823-829 (1990)), a pectin decomposition product and so forth. These may have a peculiar odor or may require that they must be added in a high concentration to show sufficient bactericidal effect.

Accordingly, a bactericide for food that comprises a safe substance, is odorless and functions at a low concentration is demanded.

It has been known that alpha-type thionin and beta-type thionin, which are peptides derived from wheat and barley, have effects of inhibiting the growth of fungi and yeast. However, few reports have been made on their effect of inhibiting the growth of bacteria. E. coli is insensitive to wheat beta-thionin since the 50% inhibitory concentration is 250 μg/mL (The Journal of Biological Chemistry, Vol. 267, pages 2228-2233 (1992)).

On the other hand, it has been reported that ethylenediaminetetraacetic acid (EDTA) has a chelating action on various metal ions and that it shows antibacterial activity against E. coli and V. parahaemolyticus in a high concentration (10 mM) (Japanese Journal of Bacteriology, Vol. 47, pages 625-629 (1992)). 10 mM disodium salt of EDTA is calculated as 3362 ppm. However, no antibacterial effect against cariogenic bacteria or periodontal bacteria has been known.

Still further, EDTA and metal salts thereof have a color stabilizing effect on food so that they are used as a food additive in USA, etc. However, their acceptable concentration is 36 to 500 ppm for disodium salt of EDTA (Food and Chemical Toxicology, Vol. 38, pages 99-111 (2000)). Therefore, even if EDTA and metal salts thereof were used alone as a food preservative in their safe concentration range, no antibacterial effect could be expected.

### SUMMARY OF THE INVENTION

Therefore, an object of the present invention is to provide a bactericidal composition that has a bactericidal effect against food poisoning bacteria at a low concentration and is highly safe.

The first aspect of the present invention relates to a bactericidal composition comprising as effective ingredients (a) at least one substance selected from the group consisting of ethylenediaminetetraacetic acid (sometimes referred to as EDTA hereinafter) and metal salts thereof and (b) at least one substance selected from the group consisting of alpha-type thionin and beta-type thionin.

The second aspect of the present invention relates to a bactericidal composition according to the first aspect of the present invention, in which the bactericidal composition is a bactericidal composition for food poisoning bacteria.

The third aspect of the present invention relates to a bactericidal composition according to the second aspect of the present invention, in which the content of the above (a) at least one substance selected from the group consisting of EDTA and metal salts thereof is 0.05 mM or more and less than 1.5 mM and the content of the above (b) at least one substance selected from the group consisting of alpha-type thionin and beta-type thionin is 1 μg/mL or more and 150 μg/mL or less.

### BRIEF DESCRIPTION OF THE DRAWINGS

FIG. 1 is a graph illustrating minimum bactericidal concentration (MBC) of alpha-type thionin of wheat when food poisoning bacteria are cultivated at 37° C. for 1 day in an LB medium containing EDTA-2Na in a varied concentration.

### DETAILED DESCRIPTION OF THE INVENTION

The bactericidal composition of the present invention contains at least one substance selected from the group consisting of EDTA and metal salts thereof and at least one substance selected from the group consisting of alpha-type thionin and beta-type thionin as effective ingredients.

EDTA binds to various metal ions. Any metal salt of EDTA may be used as far as it has an objective antibacterial activity. Suitable metal salts of EDTA include, for example, sodium salt, potassium salt and so forth. In particular, disodium salt, tetrasodium salt, and so forth of EDTA are preferred.

EDTA and its metal salts may be used alone or two or more of them may be used in combination.

Thionins, which are usable in the present invention, can be obtained from flour of grains such as barley, wheat, oats and rye through extraction with aqueous sodium chloride solution or acids such as hydrochloric acid, citric acid, etc. Alternatively, thionins can be produced by use of a genetic recombinant microorganisms or plants containing thionin genes. Both alpha-type and beta-type thionins are featured by being composed of about 45 amino acids, which contain about 8 cysteine residues, have the molecular structure characterized in that partial or every cysteine residues thereof form cross-linking by the disulfide bond or bonds.

Thionins can be purified by concentrating the extract of barley or wheat through salting out with ammonium sulfate, etc., followed by high-performance liquid chromatography or the like. However, even crude purified products obtained in the course of such purification process may be used in the present invention as far as they have an antibacterial activity. The alpha-type and beta-type thionins may be used alone or in combination with each other.

One example of the process of extracting and purifying thionins from barley is described in Planta, Vol. 176, pages 221-229 (1988). In the case where thionins are extracted from other varieties of wheat and barley and purified, this process can be applied.

There have already been known the entire amino acid sequences of thionins from barley, wheat and oats (Plant Molecular Biology, Vol. 26, pages 25-37 (1994)) and the amino acid compositions of thionins of rye (Journal of Agricultural and Food Chemistry, Vol. 26, pages 794-796 (1978)). Depending on the variety of the barley or wheat, there may also exist peptides that have substitution, addition or deletion of one or several amino acid residues in comparison with the known amino acid composition or sequence. The thionins that can be used in the present invention may also include these peptides as far as they have the objective antibacterial activity.

EDTA and its metal salts have a color stabilization effect for food and hence they are granted as a food additive in many countries.

In addition, the safety evaluation of EDTA and the like has already been established. ADI (Acceptable Daily Intake) of EDTA or the like as food additive is 2.5 mg/kg body weight (Food and Chemical Toxicology, Vol. 38, pages 99-111 (2000)).

In USA, it is allowed to add EDTA disodium (EDTA-2Na) in a concentration range of 36 to 500 ppm to food (Food and Chemical Toxicology, Vol. 38, pages 99-111 (2000)).

In consideration of rapid decomposition of thionins by a digestive enzyme such as trypsin (Journal of the Japanese Society for Food Science and Technology, Vol. 47, pages 424-430 (2000)), thionin may have a very little, if any, influence on enterobacteria. Furthermore, it has been reported that when guinea pig was administered once with 103 to 229 mg/kg body weight of thionin and observed for 7 days after the administration, no abnormality was observed (Cereal Chemistry, Vol. 19, pages 301-307 (1942)).

The bactericidal composition of the present invention can be applied to various noxious microorganisms, for example, food poisoning bacteria, cariogenic bacteria, periodontal bacteria and so forth.

The bactericidal composition of the present invention may be formulated into various forms. For example, EDTA or its metal salt and thionin may be simultaneously added to various foods. EDTA and its metal salts as well as thionins have heat resistance, so that they may be added to food prior to pasteurize the food.

To sterilize food poisoning bacteria contained in food, it may be advantageous to simultaneously add 0.1 to 0.5 mM EDTA or its metal salt and 2 to 100 μg/mL thionin for V. parahaemolyticus, 0.1 to 1.49 mM EDTA or its metal salt and 2 to 100 μg/mL thionin for Salmonella sp., or 0.5 to 1.49 mM EDTA or its metal salt and 10 to 100 μg/mL thionin for E. coli. However, the lower the concentration of EDTA or its metal salt is, the higher the concentration of thionin to be added must be.

It should be noted that 0.1 to 1.49 mM EDTA-2Na corresponds to 33.6 to 500 mg/L EDTA-2Na and is identical to or less than 500 ppm, which is the upper limit of the safety standard in U.S.A. However, there is a fear that side effects could occur when 1.50 mM or more EDTA-2Na is added to food.

According to the present invention, there is provided a bactericidal composition comprising as active ingredients (a) at least one substance selected from the group consisting of EDTA and metal salts thereof and (b) at least one substance selected from the group consisting of alpha-type thionin and beta-type thionin.

The ingredients (a) and (b) exhibit sterilizing effects at low concentrations so that food poisoning bacteria can be effectively sterilized by addition of the bactericidal composition of the present invention to various foods.

Aqueous solutions of EDTA or its metal salts and of thionin are each colorless, transparent and odorless, so that they give no influence on the taste of food and so forth.

### EXAMPLE

Hereinafter, the present invention will be illustrated in detail by examples. However, the present invention is not limited thereby.

### Production Example 1

300 mL of distilled water was added to 100 g of powdered grains of hull-less barley “Ichibanboshi”, a kind of barley, powdered by use of a cyclone mill. After stirring it at 4° C. for 1 hour, the mixture was centrifuged and the supernatant was removed.

To the precipitate was added 200 mL of 1 M aqueous sodium chloride solution and the mixture was stirred at 4° C. for 2 hours and then centrifuged. The obtained supernatant was subjected to ammonium sulfate (50 to 90% saturation), and the recovered precipitates were suspended in phosphate buffer and centrifuged. The obtained supernatant was subjected to high performance liquid chromatography to obtain purified alpha-type and beta-type thionins of barley. Using Wakosil 5C4-200, 4.6 mmφ×250 mm (manufactured by Wako Pure Chemical Industries, Ltd., Osaka, Japan) as the column, concentration gradient elution with water (pH 2.1) containing 0.1% trifluoroacetic acid and 0 to 40% (0 to 40 minutes) acetonitrile was performed at a flow rate of 0.5 mL/minute. The collected fractions were concentrated to dryness by use of a centrifugal evaporator and confirmed to be alpha-type and beta-type thionins by amino acid analysis and mass spectrum analysis. There were obtained 37 mg of alpha-type thionin and 12 mg of beta-type thionin per 1 kg of powdered “Ichibanboshi” grain.

### Production Example 2

300 mL of 0.15 N hydrochloric acid was added to 100 g of commercially available soft wheat flour and the mixture was stirred. After standing at 37° C. for 30 minutes, the mixture was again stirred and then centrifuged. To the supernatant was dropped 10 N aqueous sodium hydroxide solution to neutralize it, and centrifuged again.

The supernatant thus obtained was subjected to ammonium sulfate (50 to 90% saturation) and the recovered precipitate was suspended in phosphate buffer. The centrifuged supernatant was subjected to high performance liquid chromatography under the same conditions as in Production Example 1 to obtain purified alpha-type thionin of wheat.

The collected fractions were concentrated to dryness by use of a centrifugal evaporator and confirmed to be alpha-type thionin by amino acid analysis and mass spectrum analysis. Thus 40 mg of alpha-type thionin per 1 kg of soft wheat flour was obtained.

### Example 1

Bacillus cereus was inoculated into a sensitive measurement medium (manufactured by Nissui Pharmaceutical Co., Ltd., Tokyo, Japan), and other bacteria such as food poisoning bacteria were inoculated into an LB medium (1% polypeptone, 0.5% yeast extract, 0.5% sodium chloride, pH 7.2) and incubated at 37° C. for 1 day.

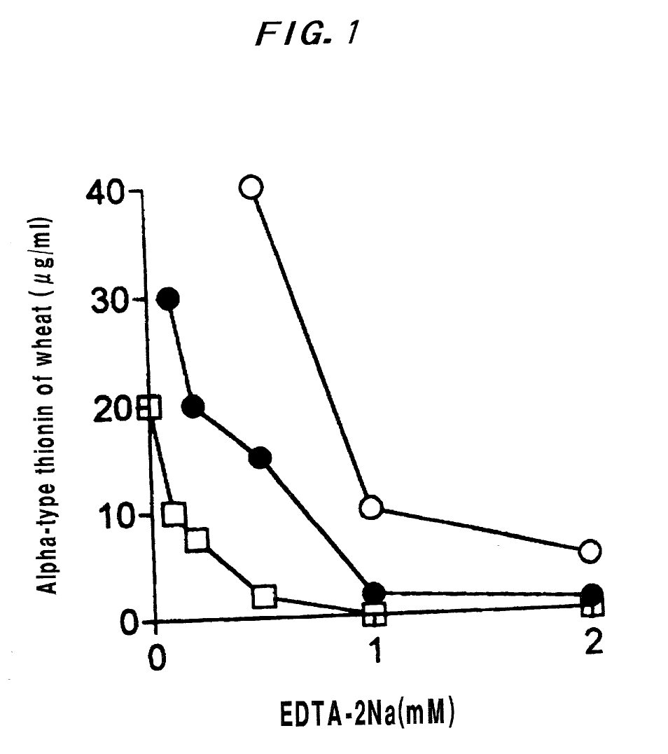

Then, after adding predetermined amounts of thionin and EDTA, the bacteria were inoculated so that the concentration of bacteria was about 1×10⁶ cells/mL. After cultivating the bacteria for 1 day for other bacteria, the viable count was measured by a method of diluted plate culture. The concentration in which the viable count was decreased to 1/100 or less was defined as minimum bactericidal concentration (MBC) and the concentration in which the viable count was not increased was defined as minimum inhibitory concentration (MIC). The results obtained are shown in Tables 1, 2 and 3, as well as in FIG. 1.

As will be clear from Table 1, when alpha-type thionin of wheat was added alone, the test bacteria except for Salmonella typhimurium JCM 6977 and V. parahaemolyticus IFO 12711 showed an MIC of 50 μg/mL or more. On the other hand, when EDTA-2Na was added alone, Salmonella sp. and E. coli had an MBC of 10 mM or more whereas other bacteria had an MBC of 1 mM or less.

Next, 10 μg/mL of alpha-type thionin of wheat and 0.02 to 10 mM of EDTA-2NA were added simultaneously. In the case of food poisoning bacteria, the MBC of EDTA-2Na was decreased as compared with the addition of EDTA-2Na alone.

Also, in the case of EDTA and EDTA-tetrasodium salt (EDTA-4Na), synergistic effects on S. typhimurium JCM 6977 and E. coli JCM 5491 identical to or higher than that of EDTA-2Na were observed (cf. Table 2).

Furthermore, the MBC of alpha-type thionin of wheat on S. typhimurium JCM 6977, E. coli JCM 5491 and V. parahaemolyticus IFO 12711 in the presence of EDTA-2Na in different concentrations was examined, and the results as shown in FIG. 1 were obtained. The tests were carried out by cultivation in an LB medium at 37° C. for 1 day. In FIG. 1, the symbol ● indicates S. typhimurium JCM 6977, ◯ indicates E. coli JCM 5491 and □ indicates V. parahaemolyticus IFO 12711. Concerning the antibacterial activity of thionin in the coexistence of EDTA-2Na, substantially no difference was observed between the thionin derived from wheat and the thionin derived from barley, and also between the alpha-type and the beta-type (Table 3).

TABLE 1

| | MIC | MBC | |
|---|---|---|---|
| | Thionin*¹ | EDTA-2Na | Thionin + |
| Strain | (μg/mL) | (mM) | EDTA-2Na*² |
| Salmonella typhimurium | 30 | 20 | 1 |
| JCM 6977 | | | |
| Salmonella enteritidis | 100< | 50< | 1 |
| IFO 3313 | | | |
| Escherichia coli 01:K1:H7 | 100< | 50 | 10 |
| JCM 1649 | | | |
| Escherichia coli 06 | 100 | 10 | 1 |
| JCM 5491 | | | |
| Vibrio parahaemolyticus | 20 | 1 | 0.1 |
| IFO 12711 | | | |
| Bacillus cereus | 100 | 0.5 | 0.02 |
| JCM 2152 | | | |
| Bacillus cereus | 100< | 0.2 | 0.05 |
| IFO 15305 | | | |

*¹Alpha-type thionin of wheat
*²MBC (mM) of EDTA-2Na in the coexistence of 10 μg/mL of wheat alpha-type thionin

TABLE 2

| | Thionin* | MBC (nM) | | |
|---|---|---|---|---|
| Strain | (10 μg/mL) | EDTA | EDTA-2Na | EDTA-4Na |
| S. typhimurium | − | 5 | 20 | 50< |
| JCM 6977 | + | 0.1 | 1 | 1 |
| E. coli | − | 5 | 10 | 10 |
| JCM 5491 | + | 1 | 1 | 1 |

*Alpha-type thionin of wheat

TABLE 3

| | | The MBC of thionin* (μg/mL) | | |
|---|---|---|---|---|
| | | Alpha-type | Alpha-type | Beta-type |
| Strain | | of wheat | of barley | of barley |
| S. typhimurium | JCM 6977 | 2 | 2 | 2 |
| E. coli | JCM 5491 | 10 | 10 | 15 |

*LB medium containing EDTA-2Na (1 mM) was used.

What is claimed is:

1. A bactericidal composition comprising as effective ingredients (a) at least one substance selected from the group consisting of ethylenediaminetetraacetic acid and metal salts thereof and (b) at least one substance selected from the group consisting of alpha-type thionin and beta-type thionin.

2. The bactericidal composition according to claim 1, wherein the bactericidal composition is a bactericidal composition for food poisoning bacteria.

3. The bactericidal composition according to claim 2, wherein the content of said (a) at least one substance selected from the group consisting of ethylenediaminetetraacetic acid and metal salts thereof is 0.05 mM or more and less than 1.5 mM and the content of said (b) at least one substance selected from the group consisting of alpha-type thionin and beta-type thionin is 1 μg/mL or more and 150 μg/mL or less.
